(19) United States
(12) Patent Application Publication
(10) Pub. No.: US 2004/0158229 A1
(43) Pub. Date: Aug. 12, 2004

(54) Catheter assembly and method of catheter insertion

(76) Inventor: David G. Quinn, Grayslake, IL (US)

Correspondence Address: Brinks Hofer Gilson & Lione, P.O. Box 10395, Chicago, IL, 60610, US

(21) Appl. No.: 10/764,674

(22) Filed: Jan. 23, 2004

Related U.S. Application Data
(63) Continuation-in-part of application No. PCT/US03/02347, filed on Jan. 24, 2003.
(60) Provisional application No. 60/351,698, filed on Jan. 24, 2002.

(51) Int. Cl. A61M 31/00

(52) U.S. Cl. 604/516; 604/528

(57) ABSTRACT

A catheter and stylet assembly in one form of the invention includes a catheter sub-assembly, a primary stylet sub-assembly and a secondary stylet sub-assembly. The dual stylet assembly facilitates individual manipulation of the stylets to vary catheter tube stiffness as the tube is inserted through the patient's nose, stomach and duodenum into the jejunum by a method of the invention. A catheter stylet and guide wire assembly in another form of the invention includes a catheter sub-assembly, a stylet sub-assembly and a guide wire. Guide wire and stylet manipulation faulitates varying catheter tube stiffness to obtain similar results. A section of the catheter tube may be normally coiled, whereby it can be straightened for insertion into the jejunum and then permitted to uncoil in the jejunum where it serves as a peristalsis-aided anchor for the tube tip.

### RELATED APPLICATION

This application is a 35 U.S.C. 111(a) continuation-in-part of PCT application No. U.S. 03/02347, filed Jan. 24, 2003, in the USPTO receiving office and designating the United States PCT application is, in turn, a continuation-in-part of U.S. provisional application Serial No. 60/351,698, filed Jan. 24, 2002. This application claims priority from the U.S. designated application and from the provisional application.

### FIELD OF THE INVENTION

This invention relates generally to catheters and catheter insertion methods. It relates particularly to catheter, stylet and guide wire assemblies for introducing a catheter into a patient's body cavity. It also relates to methods of inserting catheters into a patient's jejunum.

### BACKGROUND OF THE INVENTION

Stylets are used in medical practice situations where the clinician desires to push the catheter into a body cavity or vessel. Naso-enteral feeding tubes are inserted with stylets, as are Groshong intravenous catheters. Stylets have constant stiffness over their entire length, however. This can be a disadvantage when the catheter must travel around acute angle bends or through valves or sphincters. Having the stylet constructed with a softer leading portion can be a disadvantage because in its course of travel the catheter may encounter obstructions that will not readily permit the entry of the leading softer portion of the stylet.

Guide wires, on the other hand, are used where the clinician simply wishes to guide a catheter into a body cavity. A naso-enteral feeding tube, for example, might be guided into a patient's stomach over a guide wire.

Neither presently known stylet or guide wire assemblies or systems are effectively utilized where jejunal insertion is desired. This is particularly problematical at the present time because there is an increasing interest in placing naso-enteral feeding tubes into the jejunum, rather than the stomach or duodenum. Clinical data shows that in long term (one week or more) nasal-enteral feeding the incidence of aspiration and reflux of feeding formula into the lungs is more than 20%. This reflux occurs because the duodenum has weak peristalsis and is subject to retrograde (reverse) peristalsis that tends to push feeding formula and/or feeding tubes back into the stomach. On the other hand the jejunum has strong peristalsis. This strong peristalsis coupled with the added bends of the tube in the intestine aids in keeping the tube in place in the jejunum and keeping the formula isolated from the stomach.

There is also an increased understanding of the need for immediate jejunal feeding after gastric or intestinal surgery. Present practice is for the patient to be denied any oral, nasogastric or naso-jejunal feeding post surgically until peristalsis returns. It takes between one and eight days for peritalsis to return depending on the degree of gastric or intestinal insult caused by the surgery. During this period a suction tube is placed into the stomach via the nasal route to aspirate any gastric juices that build up in the stomach and present the danger of pulmonary aspiration.

Present practice is to place tubes into the jejunum by endoscopy. Patient sedation is required and gastroenterologists have difficulty pushing the tubes out of the stomach, through the pylorus and then past the Ligament of Trietz.

### SUMMARY OF INVENTION

An object of the invention is to provide a new and improved catheter and dual stylet assembly for inserting a feeding tube into a body cavity.

Another object is to provide a catheter and dual stylet assembly affording almost infinite varying stiffness adjustability over its leading distal portion.

Still another object is to provide a catheter and dual stylet assembly with variable stiffness over much of the entire length.

A further object is to provide a catheter and combination stylet and guide wire assembly for inserting a feeding tube into a body cavity.

Yet a further object is to allow the insertion of a naso-enteral tube into the jejunum by pushing the tube exteriorly, thereby eliminating the need for endoscopy.

Another object is to provide a tube insertion assembly which allows the use of a small, very flexible 8 Fr feeding tube, rather than stiffer 10 Fr or 12 fr tubes.

Still another object is to provide a catheter and dual stylet or stylet and guide wire assembly which facilitates easy insertion of the catheter into the jejunum yet prevents, through the catheter design, undesirable withdrawal of the catheter therefrom.

Another object is to prevent coiling of the tube in the stomach during the procedure of inserting it through the pylorus.

Another objective is to provide a catheter tube that creates minimal friction along its entire outside surface from the nares to its tip in the jejunum.

Another object is to prevent necrosis in the nasopharynx, pylorus and the Ligament of Trietz by allowing the use of a small, flexible 8 Fr tube.

Another object is to incorporate a stylet tip configuration that provides maximum protection against the inadvertent pushing of the stylet through the tube wall.

Another object is to provide a catheter with very lubricious water activated internal and external coating to assure both easy insertion and free movement of the stylets or stylet and guide wire within the tube.

Still another object is to provide a catheter and dual stylet or stylet and guide wire assembly of the aforedescribed character which includes a multi-lumen tube containing a gastric suction lumen.

The catheter and dual stylet or stylet and guide wire assembly of the present invention can be used for any medical catheter insertion where a stylet or guide wire is conventionally used. One embodiment described in the present application is used for naso-enteral insertion of a feeding tube into and through the stomach, the duodenum and finally into the jejunum. This use presents some unique requirements for adjustable variability of tube stiffness. Moderate stiffness is provided for initial insertion through the nasopharynx, the esophagus and into the stomach. Considerably more stiffness is then provided to transmit pushing force from outside the patient to the catheter tip as it is pushed through the pylorus into the duodenum and to the Ligament of Trietz. After the stiffened leading end of the catheter tube reaches the Ligament of Trietz, it is made more flexible so it can navigate around the tight curve formed by the Ligament of Trietz and into the jejunum.

In this jejunal insertion application, the stylet assembly of the invention provides moderate stiffness over a long, 25-35 inch distal length during gastric insertion. Much greater stiffness is then provided over the entire length of the catheter during duodenal insertion. Finally, moderate to very little stiffness is provided over 10 to 15 inches of the distal end as it enters the jejunum.

In one embodiment of the invention, an assembly has two stylets and allows the stylet wire of one to pass through the proximal connector of the other. This assembly also allows irrigation of the inside of the tube through the stylet connection of the assembly to activate a lubricious coating inside the tube lumen (which allows stylet wire manipulation and removal). The coating is also on the exterior surface of approximately the first 35 inches of the distal tube and tip to minimize friction along the surface of the tube from the insertion point at the nares along its entire length to the tip in the jejunum. The coating adheres to the urethane surface and is activated by contact with water.

The stylet that seats itself in the feeding tube Y-connector contains a stylet wire whose tip end position is most distal. This stylet is referred to as the primary stylet. A secondary stylet connects to the luer lock of the primary stylet and its tip is normally immediately adjacent the primary stylet tip.

The overall tube is approximately 60 inches long. This compares to a conventional feeding tube length of approximately 45 inches. The extra length tube provides for entry into the jejunum.

The method of insertion of a catheter tube using the dual stylet assembly embodiment of the invention comprises:

1. seating the primary and secondary stylets into the Y-connector;

2. flushing the tube with water through the secondary stylet connector to activate the lubricious coating on the entire length of the tube's internal lumen;

3. retracting the secondary stylet to a wire mark located 35 inches distal from the stylet connector;

4. dipping approximately 6 inches of the tube and tip in water;

5. inserting the tube in a conventional manner while, at the same time, passing the tube at the point of entry at the nares through a moist gauze pad to activate the external lubricant;

6. after the tube enters the stomach, pushing in and re-connecting the secondary stylet connector to the primary connector;

7. under flouroscopy, advancing the primary and secondary stylet stiffened catheter into the duodenum to the Ligament of Trietz;

8. retracting the secondary stylet to a wire mark located 12 inches distal from the primary stylet connector;

9. under flouroscopy, advancing the tube tip to its desired final location, beyond the Ligament of Trietz and in the jejunum;

10. while holding the Y-connector, retracting the secondary stylet and discarding it;

11. while still holding the Y-connector, retracting the primary stylet carefully so as to not dislodge the tube from the jejunum; and

12. securing the catheter with a bandage and beginning the feeding operation.

The method of insertion of a catheter tube using the stylet and guide wire assembly embodiment of the invention comprises:

1. inserting a stylet and a stylet connector into a Y-connector on the proximal end of a feeding tube; and connecting the stylet connector to the Y-connector;

2. flushing the tube with water through the connector to activate the lubricious coating on the entire length of the tube's internal lumen;

3. dipping approximately 6 inches of the tube and tip in water;

4. feeding the tube containing the stylet into the stomach, while moistening the exterior of the tube to activate the external lubricant;

5. inserting a guide wire through the stylet connector and feeding tube until its tip reaches the distal end of the tube, adjacent the stylet tip;

6. under fluoroscopy, advancing the guide wire through the pylorus and the duodenum, past the Ligament of Trietz and into the jejunum;

7. advancing the stylet stiffened tube through the pylorus and the duodenum, to the Ligament of Treitz;

8. pulling the stylet tip back through the pylorus;

9. under fluoroscopy, advancing the catheter tip on the wire, past the Ligament of Trietz, into the jejunum; and

10. while holding the Y-connector, retracting and discarding the wire and stylet.

In an alternate to this approach, the tube may be advanced from the stomach through the pylorus over the guide wire alone, i.e., without advancing the stylet further.

In an alternate form of the invention assemblies and methods, a multi-lumen tube with a mid-port bolus is employed. The catheter contains a suction lumen, in addition to the feeding lumen, and accesses both the stomach and the jejunum. The dual stylet or stylet and guide wire systems of the invention can also be used in this multi-lumen configuration. They then provide for both jejunal feeding and gastric suction.

Regardless of whether the aforedescribed assemblies and methods incorporate single lumen or multi-lumen tubes with mid-port boluses, or utilize dual stylet or stylet and guide wire combinations, in another alternate form of the invention a normally coiled, single lumen tube section is formed in the tube adjacent the distal end of the tube. This coiled section is then uncoiled, i.e., straightened, by a stylet and/or the guide wire and passed through the stomach, pylorus and duodenum to the Ligament of Trietz. The tip of the tube is then moved into the jejunum over the guide wire in this configuration. When the bolus tip of the tube is properly positioned in the jenunum, the guide wire is removed, permitting the section to resume its normally coiled, larger diameter configuration. This configuration enhances the effect of peristalsis in the jejunum on the tube and tip, and retains them more securely in the jejunum.

### BRIEF DESCRIPTION OF THE DRAWINGS

The invention, including its construction and method of operation, is illustrated more or less diagrammatically in the drawings, in which:

FIG. 1 is a side elevational view of a common, flow-through stylet connector;

FIG. 2 is an end view of the connector of FIG. 1;

FIG. 3 is a sectional view through the connector of FIG. 1 showing the flow-through lumen and the socket for the wire stylet;

FIG. 4 is a sectional view of the connector of FIG. 1 with a stylet in place;

FIG. 5 is a side elevational view of the assembled primary stylet and connector;

FIG. 6 is a side elevational view of a short segment of vinyl tubing;

FIG. 7 is a side elevational view of a male luer lock connector designed to fit over a delivery tube;

FIG. 8 is a side elevational view of the flow-through connector shown in FIG. 5, assembled to the male luer lock in FIG. 7 by means of both parts being inserted and glued into the short segment of vinyl tubing;

FIG. 9 is a longitudinal cross sectional view of the assembly of FIG. 8;

FIG. 10 is a side elevational view of the primary and secondary stylets connected, the tip of the secondary stylet being slightly behind the tip of the primary stylet;

FIG. 11 is a longitudinal cross sectional view of the primary and secondary stylets unconnected, but with the secondary stylet extending through the flow-through lumen of the primary stylet;

FIG. 12 shows the distal ends of both stylets' as they would be positioned in a feeding tube with both stylet connectors attached;

FIG. 13 is a sectional view through the tube in FIG. 12, at line 13-13 of FIG. 12, showing the main body of the primary stylet and through the tip of the secondary stylet, the tube being 8 Fr with an 0.080 inches internal lumen and the stylet wire having a 0.014 inch OD;

FIG. 14 is a sectional view through the tube in FIG. 12 and the main bodies of both stylets, at line 14-14;

FIG. 15 is a side elevational view of the first form of catheter assembly, with the stylet connectors assembled, but not seated, in the feeding tube Y-connector with a cross sectional view of the tube and tip showing the position of the stylets;

FIG. 16 is a view similar to FIG. 15 showing the connected two stylet assembly seated in the Y-connector with the distal tip of the primary stylet positioned just behind the tube tip;

FIG. 17 is a view similar to FIG. 15 showing the two stylet assembly with the secondary stylet retracted;

FIG. 18 is a view similar to FIG. 15 showing both stylets disconnected from each other and from the Y-connector;

FIG. 19 is a view similar to FIG. 15 showing the secondary stylet removed and the primary stylet seated in the Y-connector as it would be in a normal feeding tube insertion procedure;

FIG. 20 is a longitudinal sectional view through a tube illustrating the almost infinite stiffness adjustability of the catheter, i.e., there are three potential stiffness zones in the tube, all adjustable;

FIG. 21 shows a feeding tube during insertion in a patient with the secondary stylet retracted approximately 35 inches so that gastric insertion can be carried out with normal stylet stiffness;

FIG. 22 shows the secondary stylet re-seated in the Y-connector;

FIG. 23 shows both stylets remaining in place for maximum stiffness, the catheter having been pushed through the pylorus and into the duodenum up to the Ligament of Trietz;

FIG. 24 shows the secondary stylet retracted approximately 12 inches, the primary stylet remaining in place, so as to provide reduced but adequate stiffness so that the tube can be pushed around the Ligament of Trietz into the jejunum;

FIG. 25 shows the secondary stylet removed after final placement of the tip in the jejunum;

FIG. 26 illustrates the tube ready for infusion of nutrients after careful removal of the secondary stylet;

FIG. 27 is a side elevational view of a mid-port bolus in a multi-lumen feeding tube with the dual stylet embodiment of the invention are employed;

FIG. 28 is a top plan view of the bolus of FIG. 27;

FIG. 29 is a longitudinal sectional view taken along lines 29-29 of FIG. 27;

FIG. 30 shows a feeding tube with a stylet assembly that has been inserted into the stomach with the tube tip positioned adjacent to the pylorus;

FIG. 31 shows the stylet fitting seated in the Y connector and the distal end of the stylet immediately adjacent the bolus tip. The guide wire has been fed through the catheter assembly into the jejunum while the catheter tube and tip remain adjacent to the pylorus in the stomach;

FIG. 32 shows the catheter tube and tip after it has been pushed through the pyloric valve over the guidewire and is adjacent to the Ligament of Treitz, still on the guidewire;

FIG. 33 shows the catheter tube and tip after the stylet has been released from the Y connector and the tube has been advanced over the guide wire to its final position beyond the Ligament of Treitz;

FIG. 34 shows the catheter tube with the stylet removed;

FIG. 35 shows the catheter tube with the guide wire pulled out completely;

FIG. 36 shows a cross-section of the tip bolus with a passage for the guide wire;

FIG. 37 is a side elevational view of the distal end of a modified catheter tube and tip employed in another form of the method embodying features of the present invention, the tube incorporating a coiled section adjacent the bolus tip;

FIG. 38 is a view similar to FIG. 35 showing the tube at its distal end threaded over a guide wire whereby the coil section of the tube has been uncoiled by the stiffer wire, and a stylet inserted to a point adjacent the bolus tip; and

FIG. 39 is an enlarged view of the distal end of the tube with the bolus tip in the jejunum and the coiled section recoiled after removal of the guide wire and stylet.

### DESCRIPTION OF THE PREFERRED EMBODIMENTS

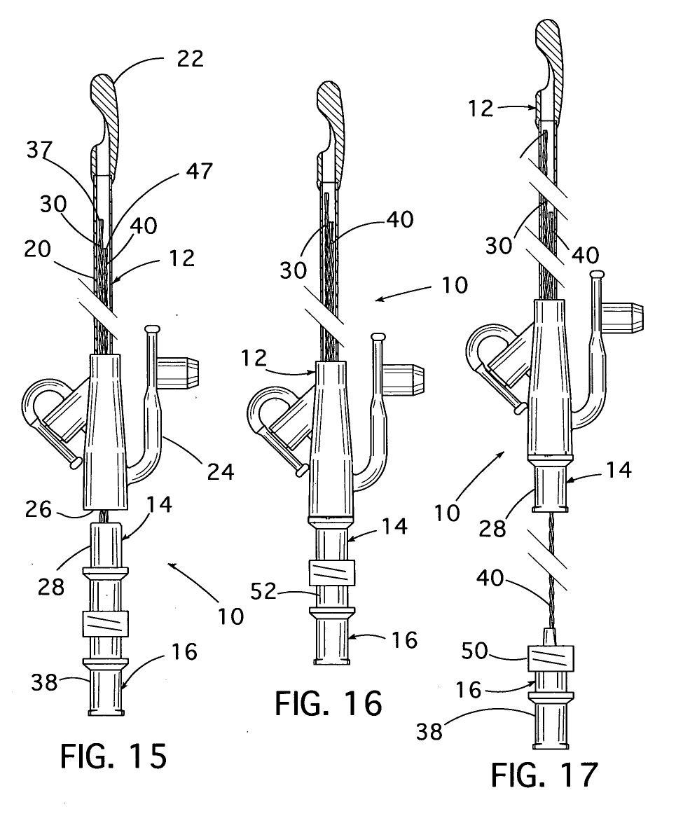

Referring to the drawings, and first to FIGS. 15-20, a catheter and stylet assembly embodying features of the invention is shown generally at 10. The assembly 10 comprises a catheter tube sub-assembly 12, a first stylet sub-assembly 14 and a second stylet sub-assembly 16.

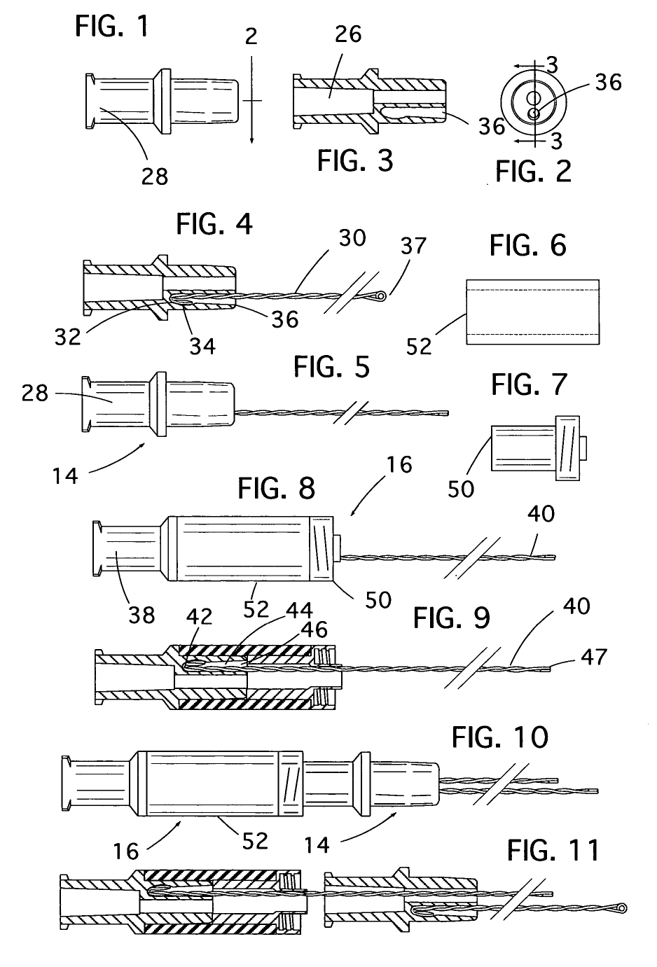

Referring now also to FIGS. 1-14, the catheter tube sub-assembly 12 comprises an 8 French (Fr) tube 20 having a bolus tip 22 on its distal end. A conventional Y-connector 24 is mounted on its proximal end. The Y-connector has a conventional inlet port 26 at its proximal end.

The first stylet sub-assembly 14 comprises a molded acrylic connector sleeve 28 and a primary twisted wire stylet 30. The proximal end 32 of the stylet 30 is force fit into a slot 34 formed within, and on the side, of a lumen 36 extending longitudinally through the connector sleeve 28. The distal end 37 of the stylet extends approximately 48 inches from the nose of the sleeve 28.

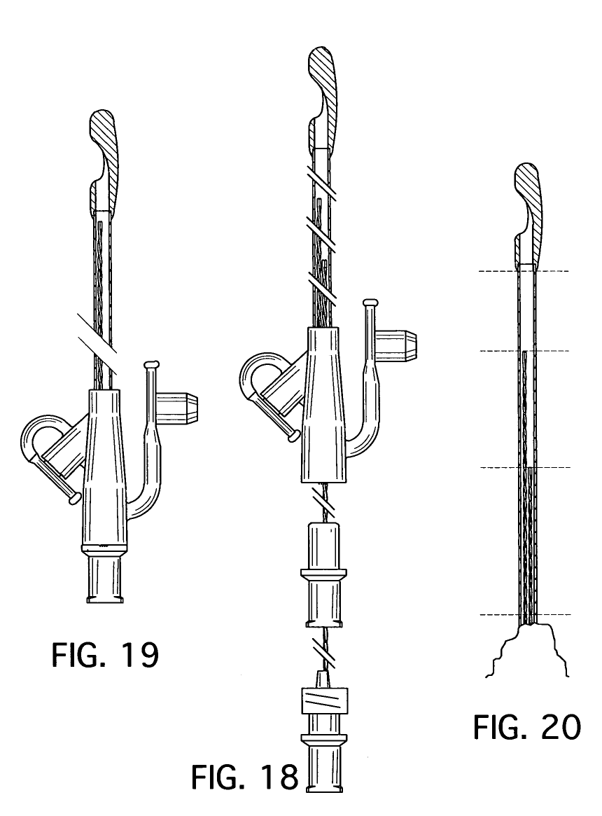

The connector sleeve 28 can be threaded into the proximal end port 26 of the Y-connector 24. Its stylet 30 then extends through the tube 20, as illustrated in FIG. 19.

The second stylet sub-assembly 16 comprises a molded acrylic connector sleeve 38 identical to the sleeve 28. A secondary twisted wire stylet 40 has a proximal end 42 which is force fit into a slot 44 formed inside, and on one side of, a lumen 46 extending longitudinally through the connector sleeve 38. The distal end 47 of the secondary stylet 40 extends approximately 52 inches from the nose of the sleeve 38.

The second stylet sub-assembly 16 also comprises an acrylic, male luer lock fitting 50 and a vinyl sleeve 52. The vinyl sleeve 52 connects the luer lock fitting 50 to the connector sleeve 38.

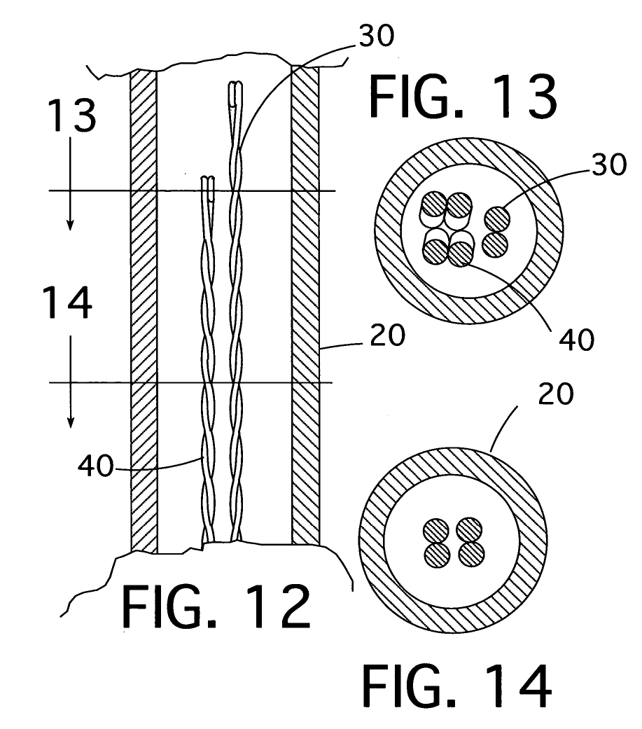

The catheter tube sub-assembly 12, first stylet sub-assembly 14 and second stylet sub-assembly 16 are joined together to form the assembly 10 in the manner illustrated in FIG. 16. In this assembly 10, the relationship of the stylets 30 and 40 are shown in FIGS. 12-14.

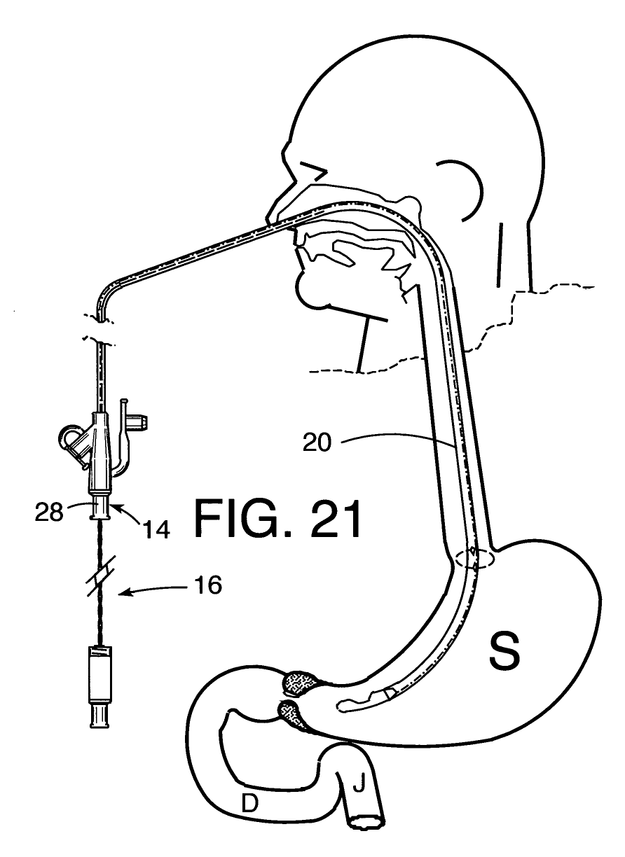

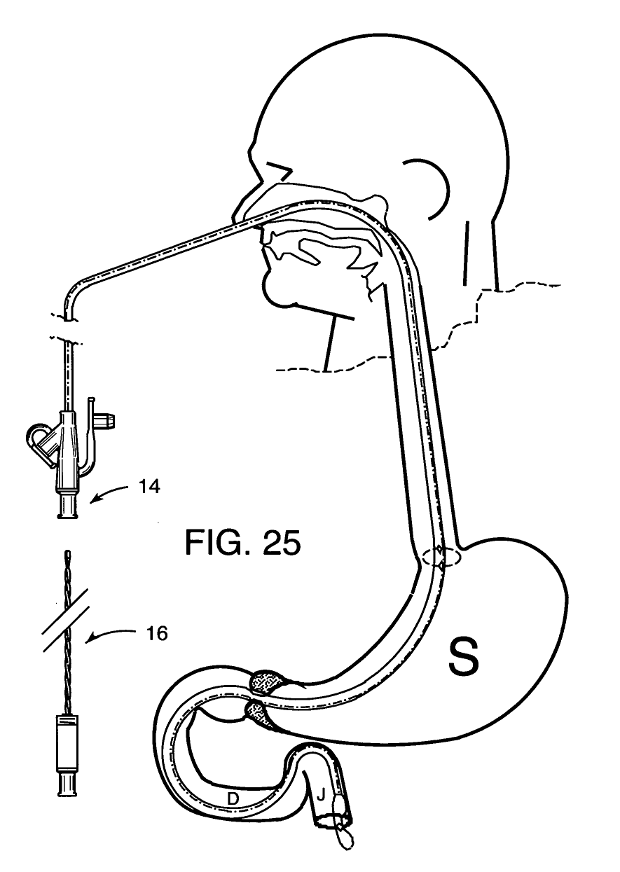

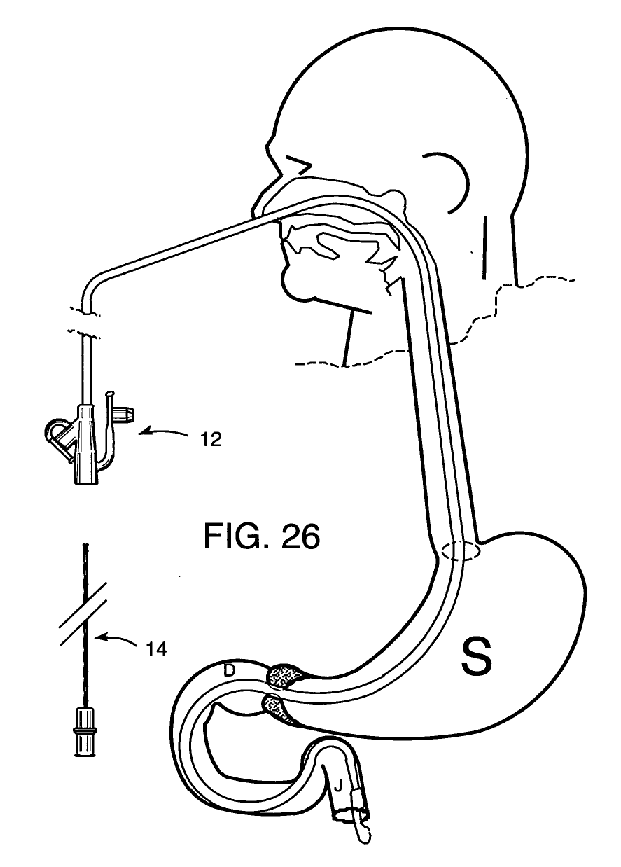

Referring now to FIGS. 21-25, FIG. 21 shows stylet sub-assembly 16 disconnected from stylet sub-assembly 14 and its stylet 28 withdrawn approximately 35 inches. In this configuration, the assembly 10 provides a more flexible tube 20 for insertion through a patient's nose.

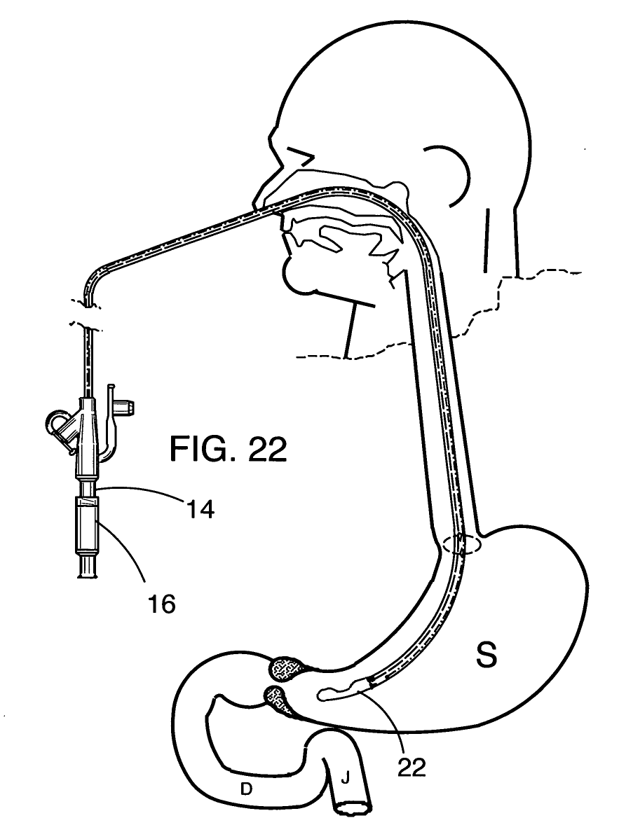

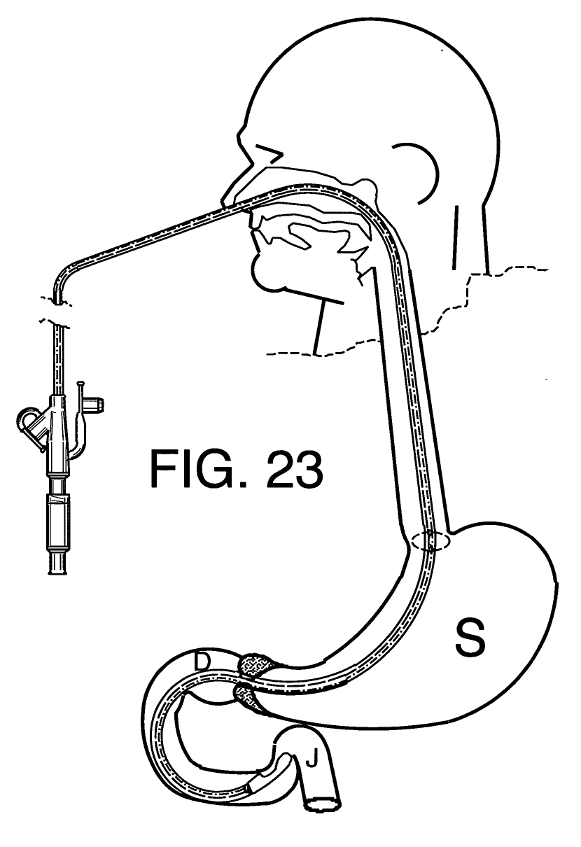

In FIG. 22, the first and second stylet sub-assemblies 14 and 16 are connected. In this configuration both stylet tips are positioned just behind tube tip 22. This facilitates passage of the tube 20 through the pyloric valve to the position shown in FIG. 23.

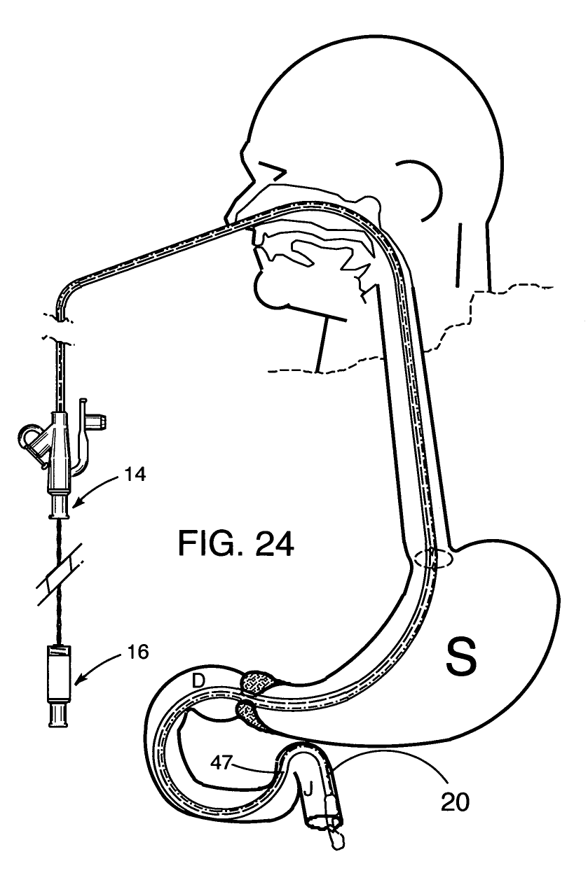

When a more flexible distal end of the tube 20 is needed, the second stylet sub-assembly 16 is disconnected from the first stylet sub-assembly 14 using the luer lock fitting. The secondary stylet can then be withdrawn a short distance (12 inches as seen in FIG. 24). This permits easy passage into the duodenum, for example.

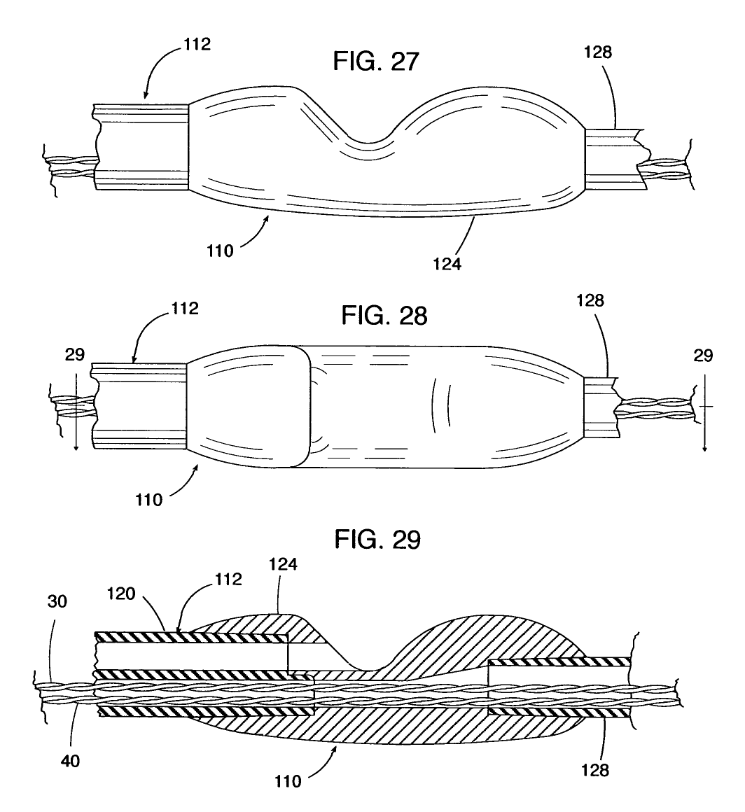

Referring now to FIGS. 27-29, a modified application of the invention is illustrated in the form of a catheter and stylet assembly 110 (with parts removed). In the assembly 110, the primary and secondary stylets 30 and 40 extend through a dual lumen catheter tube sub-assembly 112. The catheter tube sub-assembly 112 includes an 8 Fr dual lumen tube 120, a mid-port bolus 124 and a 5 Fr single lumen tube 128. The stylets 30 and 40 extend through the lumens and the mid-port bolus in the manner illustrated.

The use of a lubricious coating in the catheter insertion process has previously been described. The lubricant is a combination of methyl cellulose and polyvinylpyrolidone. The lubricant adheres to the tube surface and becomes extremely slippery when contacted by water.

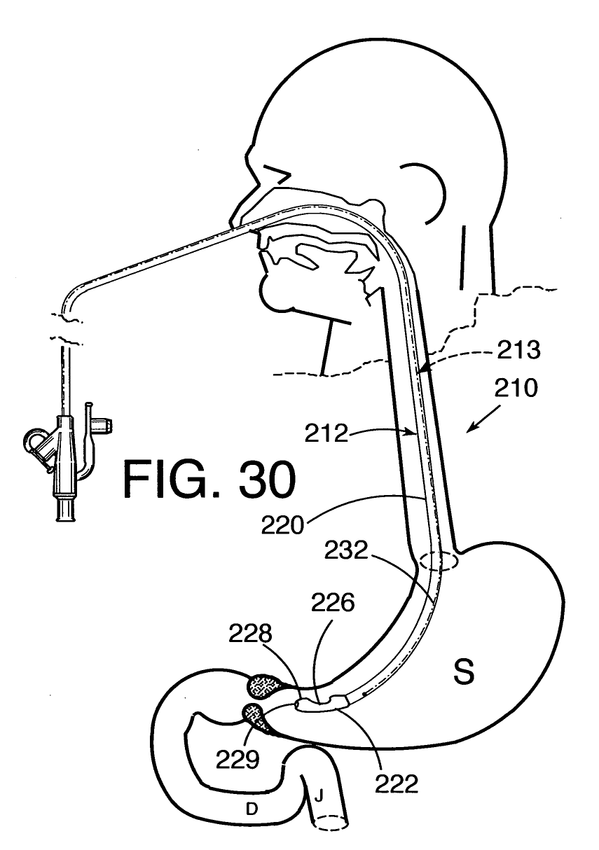

Turning now to FIGS. 30-35, a catheter, stylet and guide wire assembly embodying features of a second form of the invention is shown generally at 210. The assembly 210 comprises a catheter tube sub-assembly 212, a stylet sub-assembly 213 and a guide wire 214. The catheter tube sub-assembly includes a catheter tube 220 and tip bolus 222.

There are several ways of inserting the catheter tip 222 into the jejunum according to the invention. With a first, an ultra-slim endoscope is inserted through the nose and advanced through the stomach, into the duodenum, through the pyloric valve. The scope is advanced just proximal to the Ligament of Treitz. The guide wire 214 is then threaded through the scope and fed beyond the scope, around the Ligament of Trietz, into the jejunum. The scope is then carefully removed, leaving the guide wire in place. The catheter assembly with the stylet in place is then fed over the guide wire until the bolus tip 222 comes into proximity with the pyloric valve. The catheter tip 222 has a passage 229 formed in its bullet nose 228 that allows the passage over the guide wire. The stylet connector is then unseated from the catheter Y-connector. The stylet is held in place so that its distal end remains just proximal to the pylorus as the catheter is advanced over the guide wire until the bolus tip 222 is positioned beyond the Ligament of Treitz, in the jejunum. The guide wire is then removed and the catheter is ready for jejunal feeding.

Using a second procedure, the catheter is positioned in the stomach near the pylorus by using either a single stylet or the previously described dual stylet system. In the case of the dual stylet system, the secondary stylet is removed prior to the insertion of the guidewire. After the guidewire is threaded into the jejunum, the insertion procedure is identical to the first method described above. This second procedure is sequentially illustrated in FIGS. 30-35.

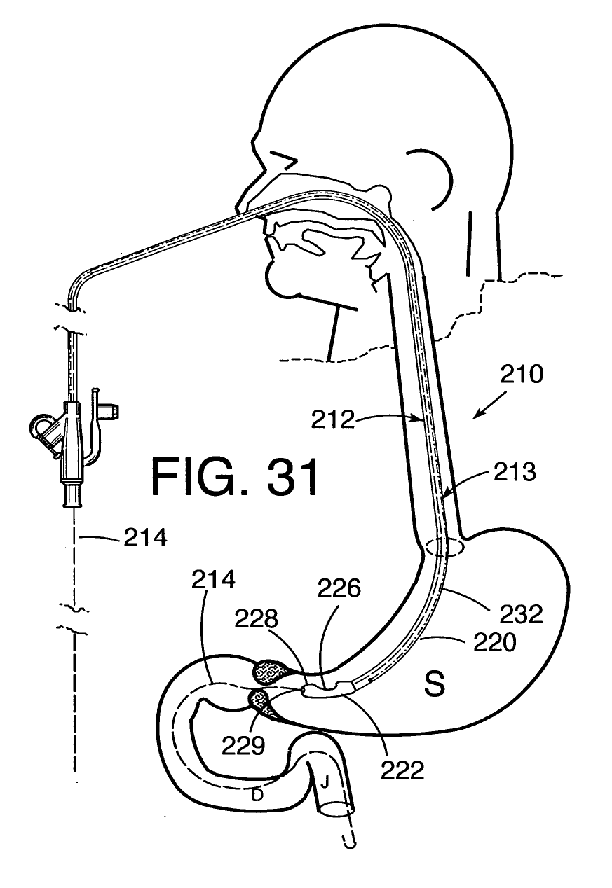

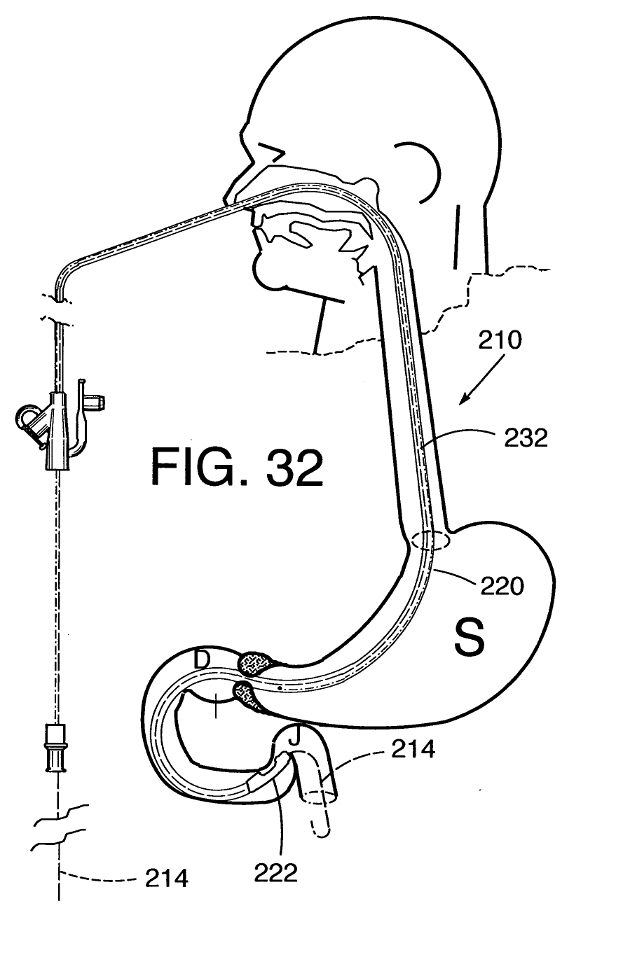

FIG. 30 shows the catheter assembly 212 in place with its tip bolus 222 next to the pylorus and a single stylet 282 in place immediately adjacent the tip bolus. FIG. 31 shows the guide wire 214 threaded through the catheter, into the duodenum and beyond the Ligament of Treitz. FIG. 32 shows the stylet connector disconnected from the catheter tube assembly and the catheter tube advanced over the guide wire 214 into the duodenum, up to a point just before the Ligament of Treitz. In the illustrated method, the distal end of the stylet remains in the stomach. However, the stylet might be advanced through the pylorus into the duodenum to provide additional stiffness to the catheter, as previously discussed.

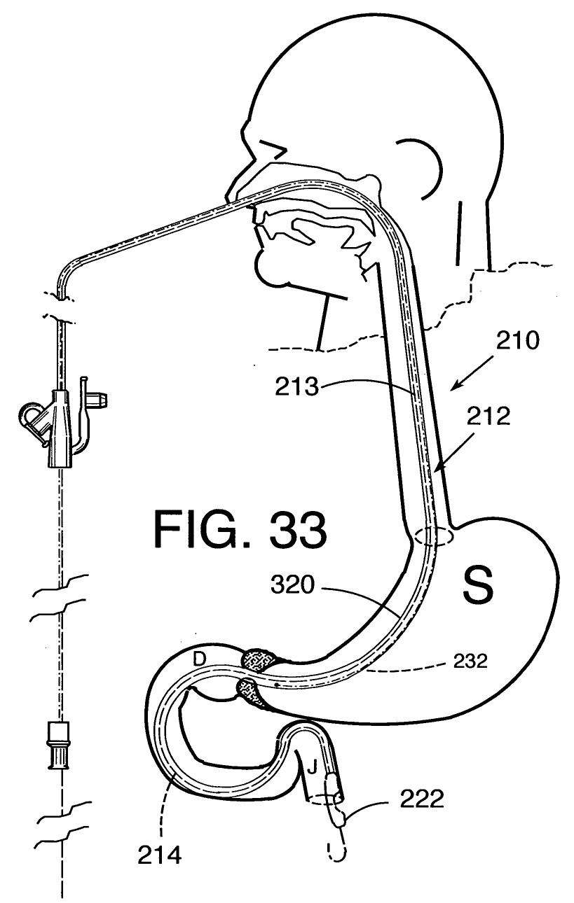

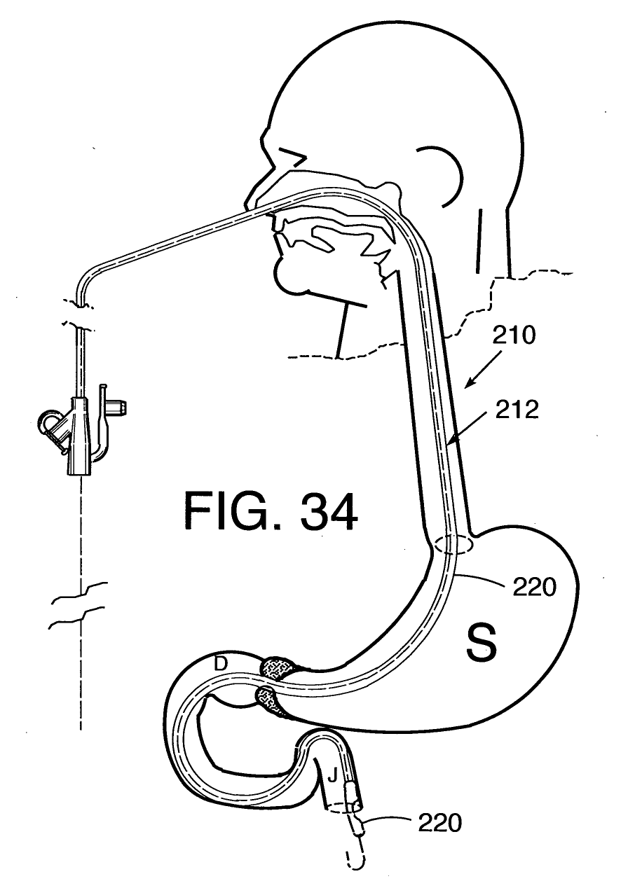

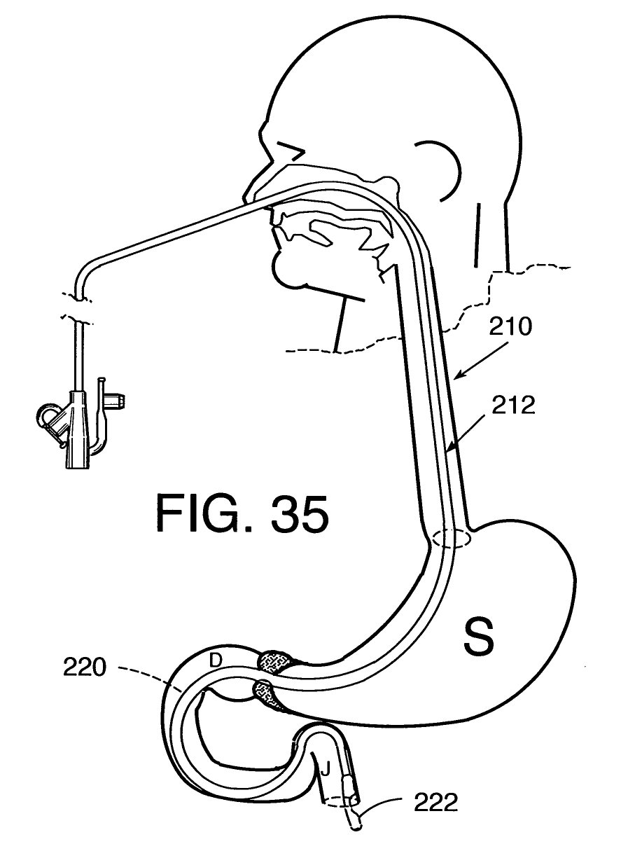

FIG. 33 then shows the catheter advanced to its final position, beyond the Ligament of Treitz and in the jejunum. FIG. 34 shows the removal of the stylet 232. FIG. 35 shows the removal of the guide wire 214. The catheter is then ready for feeding.

To this point the assembly and method inventions have been described in the context of a standard polyurethane feeding tube with a bolus on its distal end. The method invention also contemplates using a modified tube construction which enhances retention capabilities in the jejunum, however.

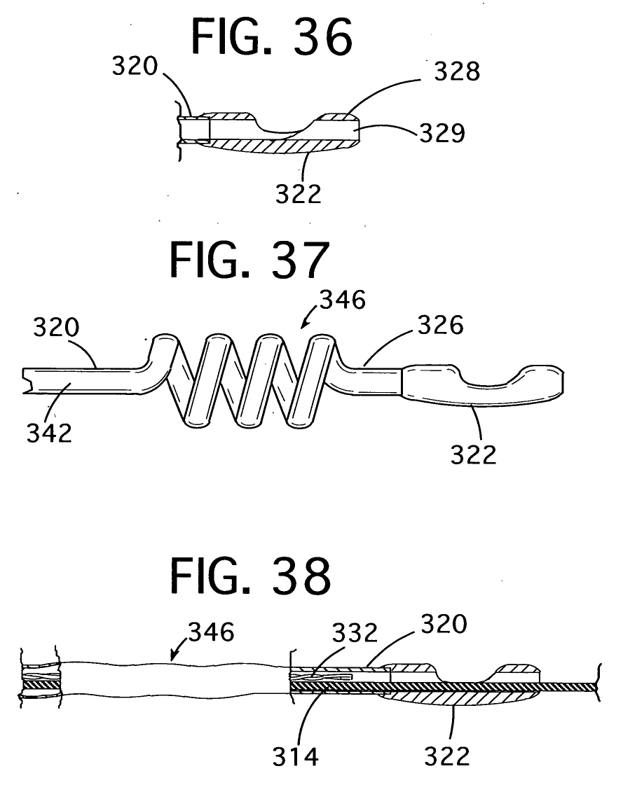

Referring to FIGS. 36-39, a catheter tube 320 is extruded to form the substantially linear section 342 extending from a conventional connector (not shown) at its proximal end to a short coil section 346 adjacent the tip bolus 322. The coil section 346 is formed after tube extrusion by the use of a jig that forms and holds the coil shape in a conventional manner while heat is applied.

The purpose of the coil section 346 is to provide a larger diameter end section on the tube that is, nevertheless, very flexible and resilient. Peristalsis exhibited by the jejunum can then hold the tip bolus 322 more easily. According to the invention, however, the coil section 336 is uncoiled, and straightened by the stylet and the guide wire before it is passed from the stomach, through the pyloric valve and into the duodenum. The passage 329 in the nose 328 of the tip bolus 322 for the wire 314 is shown.

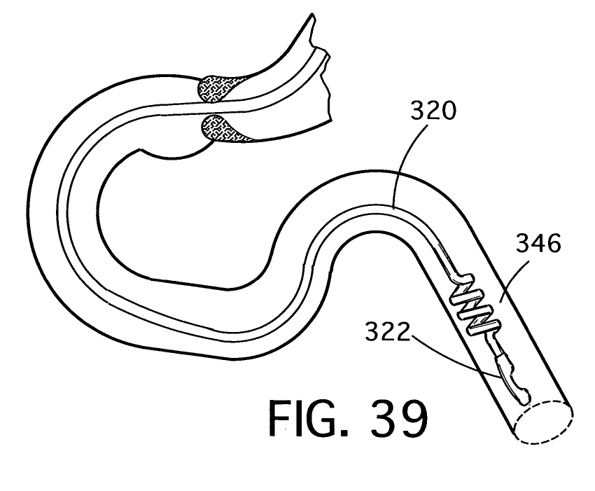

Referring now to FIG. 39, when the guide wire and stylet are removed, with the bolus 322 and the straightened coil section 346 in the jejunum, the unrestricted coil section automatically assumes its helical form. The tube 320 and tip bolus 322 then cannot be inadvertently pulled out past the Ligament of Trietz.

Thus, the tube 320 remains uncoiled and straight as long as either the stylet or the guide wire are in place, i.e., until the tip bolus 322 has entered the jejunum. Then the short coil section 336 is permitted to resume its normal shape. Enhanced retention force results.

While preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein.

1. A catheter and stylet assembly, comprising:
a) a catheter tube having a proximal end and a distal end;
b) a primary stylet extending into said tube from said proximal end; and
c) a secondary stylet extending into said tube from said proximal end;
d) said primary and secondary stylets being independently movable longitudinally of said tube to adjust the stiffness of said tube.

2. The catheter and stylet assembly of claim 1 further characterized in that:
a) said tube is an 8 Fr size tube.

3. The catheter and stylet assembly of claim 2 further characterized by and including:
a) a bolus on the distal end of said tube.

4. The catheter and stylet assembly of claim 1 further characterized in that:
a) said primary stylet includes a proximal end seated in a sleeve fitting;
b) said secondary stylet includes a proximal end seated in a sleeve fitting;
c) said primary and secondary stylet fittings being releasably connected by an intermediate sleeve fitting.

5. A stylet sub-assembly, comprising:
a) a stylet having a proximal end and a distal end;
b) a sleeve fitting having an axial passage extending longitudinally therethrough from end-to-end; and
c) a seat formed in said fitting on one side of said axial passage;
d) said proximal end of said stylet being mounted in said seat.

6. The stylet sub-assembly of claim 5 further characterized in that:
a) said seat comprises a slot formed inside said sleeve on one side of said axial passage;
b) said proximal end of said stylet being force-fit into said slot.

7. A catheter and stylet assembly, comprising:
a) a catheter tube sub-assembly including a catheter tube having a distal end and a proximal end, said tube having a connector on its proximal end; and
b) a first stylet sub-assembly including a primary stylet having distal and proximal ends, said first stylet sub-assembly also including a first stylet fitting in which the proximal end of said primary stylet is seated; and
c) a second stylet sub-assembly including a secondary stylet having distal and proximal ends, said secondary stylet sub-assembly also including a second stylet fitting in which the proximal end of said secondary stylet is seated;
d) said first stylet fitting being releasably seated in said connector with said primary stylet extending into said tube and said secondary stylet fitting being releasably connected to said first stylet fitting with said secondary stylet extending into said tube.

8. The catheter and stylet assembly of claim 7 further characterized in that:
a) said second stylet sub-assembly further including a sleeve fitting which connects said first stylet fitting to said second stylet fitting.

9. The catheter and stylet assembly of claim 7 further characterized in that:
a) said secondary stylet has a visible mark formed on it approximately 12 inches from its stylet connector.

10. The catheter and stylet assembly of claim 7 further characterized in that:
a) said catheter tube assembly includes a catheter tube containing two lumens.

11. The catheter and stylet assembly of claim 7 further characterized in that:
a) said catheter tube is a single lumen, 8 Fr size tube having a bullet nose bolus on its distal end.

12. The catheter and stylet assembly of claim 10 further characterized in that:
a) said catheter tube assembly includes an 8 Fr catheter tube containing two lumens and a smaller diameter catheter tube containing a single lumen;
b) said tubes being connected by a bolus having a side port.

13. The catheter and stylet assembly of claim 12 further characterized in that:
a) said single lumen catheter tube is a 5 or 6 Fr size tube.

14. The catheter and stylet assembly of claim 11 further characterized in that:
a) said catheter tube is coated inside and out adjacent said bolus with a water soluble lubricant.

15. A method of inserting a catheter tube into a patient's body cavity comprising the steps of:
a) providing a catheter tube having a lumen extending between a distal end and a proximal end;
b) inserting first and second stylets into said tube from said proximal end whereby said stylets are in side-by-side relationship in at least a portion of said tube;
c) introducing said catheter tube, distal end first, into an access passage to said body cavity and
d) manipulating said stylets longitudinally relative to each other in said tube to adjust the stiffness of said tube as it travels through said access passage.

16. The method of claim 15 further characterized by and including the step of:
a) coating the inside of said lumen with a water activated lubricant before inserting said stylets.

17. A method of inserting a catheter tube into a patient's jejunum comprising the steps of:
a) seating the connected primary and secondary stylets into a Y-connector;
b) flushing the tube with water through the tube to activate a lubricious coating in the tube's internal lumen;
c) retracting the secondary stylet to a wire mark located a predetermined distance distal from the stylet connector;
d) dipping approximately 6 inches of the tube and tip in water;
e) inserting the tube while, at the same time, passing the tube at the point of entry at the nares through a moist gauze pad to activate the external lubricant;
f) after the tube enters the stomach, re-connecting the secondary stylet connector to the primary connector;
g) advancing the primary and secondary stylet stiffened catheter into the duodenum to the Ligament of Trietz;
h) retracting the secondary stylet to a wire mark located a predetermined distance distal from the primary stylet connector;
i) advancing the tube tip to its desired final location, beyond the Ligament of Trietz in the jejunum;
j) while holding the Y-connector, retracting the secondary stylet; and
k) then, while holding the Y-connector, retracting the primary stylet carefully so as to not dislodge the tube from the jejunum.

18. The method of claim 15 further characterized in that:
a) said catheter tube includes a multi-lumen segment and a single lumen segment connected by a mid-port bolus.

19. A method of positioning an enteral feeding tube distal to the pylorus in a patient's gastro-intestinal system, comprising the steps of:
a) providing an enteral feeding tube having a proximal end and a distal end, said tube including a normally coiled section adjacent said proximal end;
b) inserting a stiffening element in said tube to stiffen said tube and uncoil said normally coiled section;
c) passing said distal end of said tube and said uncoiled tube section into the patient's stomach;
d) inserting the distal end of said tube past the pylorus and into the patient's duodenum with the aid of the stiffening element inside of it;
e) continuing the insertion of the tube into the patient's duodenum until the normally coiled section has entirely traversed the pylorus; and
f) pulling the stiffening element out of the proximal end of said tube to permit said normally coiled section to again assume a coiled configuration.

20. A method of inserting a catheter tube into a patient's jejunum through the nasogastric cavities, comprising the steps of:
a) inserting a guide wire through the patient's nasogastric passages, past the pyloric valve, into the duodenum and past the Ligament of Trietz into the jejunum;
b) threading a catheter tube onto the proximal end of the guide wire and manipulating the tube to cause it to follow the wire into the patient's stomach cavity;
c) inserting a stylet into the catheter tube from its proximal end until the tip of the stylet is adjacent the tip of the tube;
d) using the stylet to force the tip of the tube through the pyloric valve and duodenum to the Ligament of Trietz;
e) retracting the wire to a point where its distal end is out of the jejunum; and
f) using the stylet to force the distal end of the tube past the Ligament of Trietz and into the jejunum; and
g) withdrawing both the wire and the stylet from the tube.

21. The method of claim 20 further characterized by and including the steps of:
a) providing a catheter tube which includes a coil section adjacent the distal end of the tube;
b) threading the tube, including the coil section, over the guide wire so as to uncoil and straighten the tube in said section;
c) advancing said coil section past the Ligament of Trietz and into the jejunum while it is uncoiled; and
d) permitting the section to form a coil again in the jejunum.

22. The method of claim 21 further characterized by and including the steps of:
a) pulling said stylet out of the tube to permit the section to form a coil again.

23. A catheter assembly, comprising:
a) a catheter tube having a proximal end and a distal end;
b) a primary removable stiffening element extending through said tube from said proximal end; and
c) a secondary removable stiffening element extending into said tube from said proximal end;
d) said primary and secondary stiffening elements being independently movable longitudinally in said tube to vary the stiffness of said tube.

24. The catheter assembly of claim 23 further characterized in that:
a) said catheter tube comprises a multiple lumen tube at its proximal end and a single lumen tube at its distal end, the tubes being interconnected so that said stiffening elements can extend the full length of both tubes.

25. The catheter assembly of claim 23 further characterized in that:
a) said primary removable stiffening element is a guide wire.

26. The catheter assembly of claim 25 further characterized by and including:
a) a bolus having a bullet nose and a side port on the distal end of said tube;
b) said bolus also having an aperture in said nose through which said guide wire passes.

27. A method of inserting a catheter tube into a patient's body cavity comprising the steps of:
a) providing a catheter tube having a lumen extending between a distal end and a proximal end;
b) introducing said catheter tube, distal end first, into an access passage to said body cavity;
c) inserting first and second stiffening elements into said tube from said proximal end whereby said stylets are in side-by-side relationship in at least a portion of said tube; and
d) manipulating at least one of said stiffening elements longitudinally relative to the other in said tube to adjust the stiffness of said tube as it travels through said access passage.

28. The method of claim 27 further characterized by and including the step of:
a) coating the inside of said lumen with a water activated lubricant before inserting said stylets.

29. The method of claim 27 further characterized in that:
a) said catheter tube includes a multi-lumen segment and a single lumen segment connected by a mid-port bolus.
